(19) United States
(12) Patent Application Publication
(10) Pub. No.: US 2026/0009780 A1
(43) Pub. Date: Jan. 8, 2026

(54) METHOD OF PREPARATION OF FORMATION WATER MODEL

(71) Applicant: SCHLUMBERGER TECHNOLOGY CORPORATION, Sugar Land, TX (US)

(72) Inventors: Mikhail Reonaldovich STUKAN, Moscow (RU); Ivan Viktorovich YAKIMCHUK, Abu Dhabi (AE); Evgeny Nikolevich IVANOV, Moscow (RU); Anna Vyacheslavovna BELETSKAYA, Sugar Land, TX (US); Igor Andreevich VARFOLOMEEV, Moscow (RU); Aleksandr Sergeevich DENISENKO, Moscow (RU); Anastasia Tikhonovna REBRIKOVA, Moscow (RU)

(21) Appl. No.: 19/136,868

(22) PCT Filed: Dec. 5, 2023
(86) PCT No.: PCT/US2023/082403

§ 371 Date: Jun. 7, 2025

(30) Foreign Application Priority Data
Dec. 8, 2022 (RU) 2022132160

(51) Int. Cl. G01N 33/24

(52) U.S. Cl. CPC G01N 33/241

(57) ABSTRACT

The invention relates to methods for preparing aqueous solutions of mineral salts with a composition corresponding to that in equilibrium with solid rock minerals under the target conditions of the experiment. The technical result achieved by implementing the invention is to provide the possibility of obtaining a model of formation water with a composition closest to real formation water at target values of temperature and pressure, without the need for water sampling in the field. In accordance with the proposed method of preparation of the formation water model, a rock sample containing soluble hard skeleton minerals and/or salt deposits in the pore space, which was initially saturated with formation water and contains at least one area that has not been exposed to drilling fluid, is taken from the formation. The amount and composition of soluble sediments in the collected sample and the water saturation of the sample under reservoir conditions are determined. At least one internal region in the sample, not exposed to the drilling fluid, is selected and a three-dimensional digital model of the selected region is built, reflecting the pore structure and spatial arrangement of minerals. The water solubilization chemical reactions for all water-soluble minerals found in the sample are set. Numerical modeling of the initial saturation of the sample under study with water is carried out, taking into account the dissolution of minerals included in the sample at the required temperature and pressure until equilibrium is reached. The final ionic composition of the aqueous solution saturating the pore space of the numerical model after equilibrium is reached is determined, and the solution is prepared by dissolving the calculated amount of salts in the calculated amount of water while stirring. The resulting salt solution is brought to the required thermobaric conditions.

### CROSS-REFERENCE TO RELATED APPLICATIONS

The present application claims priority benefit of Russian Patent Application No. 2022132160, filed Dec. 8, 2022, the entirety of which is incorporated by reference herein and should be considered part of this specification.

### BRIEF DESCRIPTION OF THE DRAWINGS

FIG. 1 shows a block diagram of an embodiment of a method for producing a synthetic model of produced water according to the present disclosure.

### DETAILED DESCRIPTION

The invention relates to methods for preparing aqueous solutions of mineral salts with a composition corresponding to that in equilibrium with solid rock minerals under target experimental conditions (including formation conditions), based on data on the pore structure and mineral composition of a core sample taken from a formation of interest.

Nowadays, it is impossible to imagine planning development in a new field or making any significant changes in the development plan without preliminary modeling of intra-formation processes with the help of hydrodynamic reservoir simulators available on the market. Such simulators allow you to test different development scenarios and choose the best one. Such modeling is particularly relevant when planning secondary and tertiary modeling methods. Existing software makes it possible to reflect physicochemical processes in detail, which increases the reliability of modeling. However, the results of modeling performed using any simulator are dependent on the input parameters, with the quantity and quality of the input data being of paramount importance. The parameters that determine both the rate of hydrocarbon production from the reservoir and the potential recovery volume include capillary effects, or more precisely, the ratio of viscous and capillary forces. It is known that capillary effects are determined by the so-called interfacial tension and wettability edge angles. Interfacial tension is a measure of the affinity between immiscible fluids. Wettability is a characteristic of the interaction between the rock surface and the fluids. Consideration of formation wettability is critical in the initial stages of development planning, and improper selection of injected agents can result in reduced production, both dynamic and cumulative.

The production efficiency is significantly affected by changes in thermobaric conditions both near the wells and in the interwell space, as well as by mixing of formation water with water injected into the formation to maintain formation pressure, which can lead to precipitation, including insoluble sludge. Correct description of dynamic processes in the formation, prediction of dissolution and crystal formation is possible only with a reliable understanding of the composition of rocks and fluids saturating them.

A reliable assessment of the potential risks associated with the injection of aqueous solutions into the reservoir, as well as the measurement of rock wettability and surface tension between oil and aqueous solutions in the reservoir, requires representative conditioned rock samples and the following fluid phases: oil, injected solution and aqueous solution initially present in the reservoir. Sampling the injected water is fairly easy, but properly collecting the rest of the required samples is a challenge. However, for most wells, core or cuttings samples are available, and oil sampled at the surface can be used to some extent to create an adequate reservoir oil model (Sauerer B., Stukan M., Abdallah W., Buiting J., “Toward Determining Interfacial Tension at Reservoir Conditions Based on Dead Oil Measurements”, SPE-183671-MS, SPE Middle East Oil and Gas Show and Conference, Manama, Bahrain, 6-9 Mar. 2017).

Unfortunately, this approach is hardly applicable to formation water. Movement of aqueous solution to the surface may be accompanied by additional dissolution of minerals, mixing of solutions of productive and unproductive horizons and/or loss of some components due to their precipitation associated with changes in solubility of substances under changing thermobaric conditions. It is also worth noting that core and oil sampling techniques are more technologically advanced and much better suited to the needs of the oil industry than those for water phase sampling. In addition, the necessary data on formation water properties are often not available at the initial stage of site development. In this case, due to the lack of the necessary sample, it is necessary to rely on the availability of such data in the reports of previous years, or to use data on similar objects. Whereas core material sampled during drilling is much more readily available.

Determination of the necessary rheological properties and obtaining reliable results of laboratory work on routine and special core analysis is impossible without the use of a synthetic model of formation water corresponding to that in the reservoir. The formation water model is prepared by dissolving the salts predominant in the formation water in distilled water. However, often, due to the impossibility of obtaining a sample and/or determining the composition of formation water, a simplified formation water model is used in laboratory practice, when the total salinity is assumed to be equivalent to sodium chloride NaCl (OST 39-235-89 “Oil. Method for Determination of Phase Permeabilities in Laboratory Conditions under Joint Stationary Filtration”, p. 10). It is obvious that the use of non-equilibrium (with respect to the state of the reservoir bedding) solutions in experimental studies can lead to irreversible changes in the solid skeleton composed, for example, of evaporite minerals. In addition to the obvious framework changes due to dissolution or sedimentation processes, the ionic composition of the formation water model plays an important role in the reliability of core studies. Hydration processes characteristic of layered aluminosilicates can play a crucial role in the nature of reservoir properties of the deposit, especially for rocks with a high content of mixed-layer and swelling clay minerals, when an incorrect choice of saturating fluid dramatically changes the pore structure, up to complete destruction of rock samples at the stage of sample preparation. The acid-alkaline properties of the formation water model, which regulate the redox equilibrium of the rock-fluid system, also play an important role. Thus, the use of solutions with composition different from the equilibrium one may lead to inadequate assessment of salt deposition risks associated with changes in thermobaric conditions, contact between formation water and injected water, as well as errors in estimating mobility thresholds, residual oil saturation values and final hydrocarbon phase displacement factor.

All known approaches to obtaining water models for core studies assume as an initial stage the taking of water samples in the field and their preparation for direct use in core studies, or for conducting experiments to determine the mineral composition of water samples for the subsequent preparation of appropriate synthetic models on its basis. In this case, both variants of deep sampling, sealed or not sealed method are used (Methodological Guidelines, VNIIGAZ 1995, pp. 15-18), and variants involving surface sampling (RD 39-23-1055-84 INSTRUCTION ON METHODS OF ANALYSIS OF MINERAL CONCENTRATION OF FORMATION WATER AND SALT DEPOSITS; Methodological Guidelines for hydrogeochemical control of watering of gas and gas condensate fields. RAO GAZPROM VNIIGAZ, Moscow, 1995). Both approaches have their disadvantages. Thus, deep sampling is rather complicated and expensive, while surface sampling does not take into account possible changes in the composition of the selected sample in relation to reservoir conditions. In any case, in the absence of source formation water samples, it is not possible to obtain model formation water samples for core studies. The present invention solves this problem and makes it possible to prepare a formation water model based on the results of analyzing a rock sample without having to obtain a formation water sample.

Thermodynamic and geochemical modeling methods are widely used to determine the properties of rock solutions. However, we are not aware of approaches to reconstructing unknown formation (background) waters based on the analysis of core material using the principles of thermodynamic equilibrium between solid minerals and fluids.

The technical result achieved by implementing the invention is to provide the possibility of obtaining a formation water model with a composition closest to real formation water at target values of temperature and pressure, without the need for water sampling in the field, solely on the basis of data on the pore structure and mineral composition of rock samples taken from the formation/horizon of interest. This makes it possible to significantly accelerate acquisition and improve the quality of model formation water samples required for core studies at target values of temperature and pressure (including corresponding formation pressure).

The specified technical result is achieved by the fact that, in accordance with the proposed method of preparation of the formation water model, a rock sample containing soluble hard skeleton minerals and/or salt deposits in the pore space, which was initially saturated with formation water and contains at least one area that has not been exposed to drilling fluid, is taken from the formation.

The amount and composition of soluble sediments in the collected sample and the water saturation of the sample under reservoir conditions are determined.

At least one internal region in the sample, not exposed to the drilling fluid, is selected and a three-dimensional digital model of the selected region is built, reflecting the pore structure and spatial arrangement of minerals.

The chemical reactions of dissolution by water for all water-soluble minerals found in the sample are set and numerical modeling is carried out of initial saturation of the investigated sample with water, taking into account the dissolution of minerals included in the sample at the required temperature and pressure until equilibrium is reached.

The final ionic composition of the aqueous solution saturating the pore space of the numerical model after equilibrium is reached is determined.

The solution is prepared by dissolving the calculated amount of salts in the calculated amount of water by stirring and then bringing the resulting salt solution to the required thermobaric conditions.

According to one embodiment of the invention, the selection of an internal region of the sample that has not been exposed to drilling fluid is based on an analysis of the internal structure of the sample obtained by low resolution X-ray tomography.

According to yet another embodiment of the invention, the construction of a three-dimensional digital model of a selected region of the sample is performed based on a high-resolution X-ray micro-CT image.

Additionally, scanning electron microscopy can be used in imaging modes that provide information on the chemical/mineral composition of the sample under study and the spatial distribution of minerals.

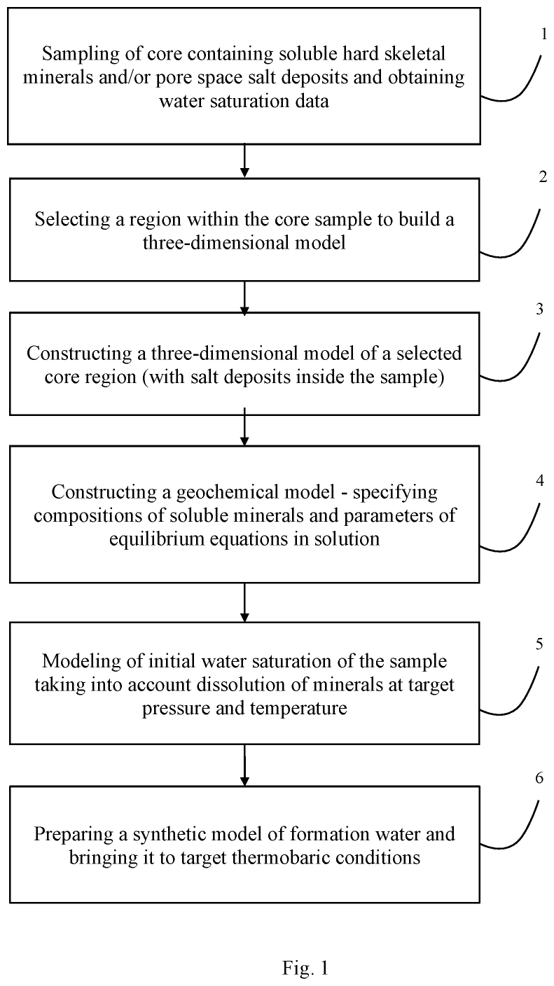

The invention is explained with reference to the drawings, wherein FIG. 1 shows a block diagram of an embodiment of a method for producing a synthetic model of produced water according to the proposed invention.

The proposed method of formation water model creation is based on determination of formation water composition by analyzing the pore structure and mineral composition of the rock sample and numerical reconstruction of the thermodynamic equilibrium in the reservoir by simulating the reverse process of water evaporation from the sample in order to determine the initial state of the considered rock-fluid system and subsequent creation of synthetic water samples corresponding to a certain composition under the required thermobaric conditions.

The method is based on the assumption that formation water and the soluble part of the mineral skeleton of the rock are physically and chemically balanced in the conditions of a formed oil-and-gas-bearing deposit. In other words, the ionic composition of formation water is formed as a result of dissolution of minerals of the contacting rocks and stabilized over geological time.

In accordance with the proposed invention, in a first step (block 1 in FIG. 1), a representative rock sample is collected from a formation of interest containing soluble hard skeletal minerals and/or pore space salt deposits. This can be core or slurry, but larger samples are preferred. A rock sample is considered suitable if it contains at least one region (volume) that was originally saturated with formation water and has not been exposed to drilling fluids. This can be either recently sampled material or taken from a core storage facility.

The sample selected for analysis should be representative of the formation/rock type under study in terms of characteristic porosity and permeability values, which may require additional laboratory tests that should be performed without altering the internal structure and composition of the sample. For example, using the gas-volumetric method.

In addition to a representative rock sample, appropriate water saturation data under reservoir conditions are also required. Such data can be obtained either directly from the sample (e.g., using the Dean-Stark method), from laboratory modeling of water saturation (drainage, capillary impregnation, etc.), or by interpreting petrophysical core and GIS data.

According to the proposed method, in a second step, at least one region in the available rock sample is selected to be used for analysis (block 2 in FIG. 1). In this case, the following basic assumption is made: after evaporation of the solution with which the core was initially (in the formation) saturated, all mineral components included in this solution are deposited in the pores of the core material. Thus, it is necessary to determine what portion of the available rock sample meets this criterion/assumption. This criterion is most likely to be met by the inner region of the rock sample that is unaffected by drilling fluid filtrate. The boundary of the drilling fluid filtrate penetration area can be determined, for example, by using contrast agents from an isolation coring tool, or selected with a margin based on available data on fluid penetration and lateral rock permeability. The initial equilibrium solution in such an area is held in place by capillary forces and is not subject to displacement due to degassing of the fluid phase and unconsolidation of the core as it rises to the surface.

The selection of an area suitable for analysis may be based, for example, on an analysis of the internal structure of the sample obtained by low-resolution X-ray tomography (e.g., sequential overview scans of a full-size core and a standard-size sample selected on the basis of the data obtained). The key criteria for selecting the area is the absence of mechanical disturbances that could potentially lead to infiltration of drilling fluid filtrate and irreversible changes in brine composition and, consequently, in the volume of skeletal soluble minerals. Unusual and unrepresentative areas of the breed should also be excluded from selection.

The method for finding a characteristic area is not governed by the present invention and may be carried out, for example, as follows. First, the entire sample is scanned using a low-resolution X-ray tomography technique (e.g., 10-100 μm/voxel). From the resulting three-dimensional image of the entire sample, the total salt content and areas containing significant amounts of soluble minerals are determined. Further, by analyzing the image, at least one region is found that is both characterized by significant overall salinization and representative of the inclusion of the full variety of soluble minerals contained in the formation. This area is subsequently scanned at a higher spatial resolution sufficient to resolve the pore space (typically less than 5 μm/voxel).

In order to increase the accuracy of the results (improve statistics), more than one characteristic area may be selected for analysis.

According to another embodiment of the invention, in order to improve the accuracy of the results (improve statistics), selection of a region of the largest possible size is carried out.

According to the proposed invention, in a third step (block 3 in FIG. 1), the construction of a three-dimensional digital twin (model) of the core region(s) selected in the second step is carried out. The digital model should correctly represent both the structure (geometry and shape) of the pores and the spatial arrangement of the various minerals, especially the distribution of evaporites and salt deposits within the sample.

In one embodiment of the invention, the three-dimensional model, is constructed from a high-resolution X-ray micro-CT image. The spatial resolution should be chosen such that the pore space geometry is resolved. Characteristic values: 0.5-5 μm/voxel. In general, X-ray microtomography does not provide information on chemical composition, but in many practical cases the method can distinguish minerals of interest. Under the condition that a small number of different phases are present, and there is a significant difference in the X-ray absorption levels of these phases, the distribution of mineral composition can be determined directly from the core micro-CT image by direct image segmentation (U.S. Pat. No. 9,927,554B2, US20140376685A1).

According to another embodiment of the invention, to build a three-dimensional model, in addition to X-ray tomography, scanning electron microscopy is used in imaging modes that provide information on the chemical/mineral composition of the sample under study and the spatial distribution of minerals. An example of such an approach is described in WO2017039475A1.

In accordance with the proposed method, the fourth step (block 4 in FIG. 1) for each mineral determined from the analysis of tomographic images or results of scanning electron microscopy sets the dissolution reactions with water in order to determine the composition of the aqueous solution in a state of equilibrium, which implies the creation of a geochemical model—the determination of equations describing the equilibrium of dissolved minerals with the rock, and their parameters. Such parameters can be values of equilibrium constants, as well as values of activity coefficients for each of the components formed in the system “mineral—aqueous solution”. The method for determining the values of equilibrium constants under given thermobaric conditions and the values of activity coefficients is not governed by this invention. For example, one can use tabulated values or derive them from basic computational chemistry or using one of the available chemical simulators (for example, Oli Studio https://www.olisystems.com/oli-studio-scalechem or Scale Soft Pitzer https://bcc.rice.edu/node/47, Fan C. et al. Scale prediction and inhibition for oil and gas production at high temperature/high pressure, SPE journal, 2012, volume 17, Nr. 02, p. 379-392). This information is needed to determine the composition of the aqueous phase in equilibrium with the rock minerals and the precipitation of salts precipitated in the pores.

According to the proposed invention, in the fifth step (block 5 in FIG. 1), numerical modeling (recovery) of the initial saturation of the test sample with water is performed. This is done by simulating the saturation of the test sample with pure deionized water, free of any impurities, at reservoir temperature and pressure until the water saturation value determined at the first stage is reached. The method of modeling is not governed by the present invention. For example, it can be performed using hydrodynamic modeling at the pore level based on density functional (Fundamentals of the density functional method in hydrodynamics// Demyanov A. Y., Dinariev O. Yu, Evseev N. V.-M.: PHYSMATLIT, 2009) or, for example, by iterative balancing of volumes of initial and final reaction components in the modeled pore volume under given thermobaric conditions, taking into account the establishment of chemical equilibrium at each iteration, using computational chemistry approaches or using one of the available chemical simulators (for example, Oli Studio https://www.olisystems.com/oli-studio-scalechem or Scale Soft Pitzer https://bcc.rice.edu/node/47, Fan C. et al. Scale prediction and inhibition for oil and gas production at high temperature/high pressure, SPE journal, 2012, volume 17, Nr. 02, p. 379-392). The composition of the solution saturated with dissolved minerals is determined by methods of chemical thermodynamics within the framework of the chosen geochemical model. Once the composition of the equilibrated solution has been determined, it is possible to estimate the amount of dissolved minerals and the resulting free volume. It is necessary to compensate for the increase in porosity by adding an appropriate amount of water to maintain reservoir pressure. After several such iterations, the model system arrives at equilibrium. Thus, both reactants (solid minerals) and products (dissolved minerals, ions in water) can be in the equilibrium system. The ionic composition of the solution in the pores thus established corresponds to the initial composition of formation water.

According to one embodiment of the invention, in order to increase the reliability of the results, saturation of the sample is carried out uniformly throughout the pore space of the digital model.

According to another embodiment of the invention, saturation of the sample is carried out by simulating non-uniform contact with the injected water when water is injected in different directions.

The quality of the data obtained can be improved by repeating steps 1-5 for several rock samples of the deposit of interest and/or for different areas of the same sample and averaging the results obtained.

After determining the equilibrium composition of the formation water in the sixth step (block 6 in FIG. 1) of the invention, a synthetic model of the formation water is created by dissolving the mineral salts, the composition and amount of which were determined in steps 1-5, in an appropriate amount of pure deionized water (also determined in steps 1-5). The quantities of mineral salts and water are pre-weighed before the dissolution procedure is carried out. The resulting solution is then brought to the required thermobaric conditions—target temperature and pressure.

As an example, consider the preparation of a synthetic model of produced water with a composition corresponding to the composition of produced water from an aquifer, where water saturation is 100%. The formation is composed predominantly of sandstone (and binder insoluble cement).

The formation conditions correspond to a temperature of 30° C. and a pressure of 30 MPa.

According to the present invention, in a first step, a rock sample was collected, and laboratory measurements were taken. The rock sample is a cylindrical core measuring 30 mm in diameter and 30 mm in height. The external volume was determined by measuring the geometric dimensions of the specimen using a laser caliper. The measurement of geometric dimensions was performed at three points and averaged to reduce the determination error. The sample was dried until mass constancy was achieved for 72 hours at 105° C.

The mineral volume of the sample was measured by gas-volumetric method (helium) under atmospheric conditions. The open porosity was determined from the two volumes by bringing the difference between them to the outer volume of the sample Laboratory mineralogical studies of the rock sample, conducted on its end sections, allow us to conclude that the insoluble mineral matrix consists mainly of quartz (more than 90% of the volume), and insoluble and poorly soluble calcareous cements-gypsum, dolomite and anhydrite (about 3% in total). The remaining amount is made up of minerals that can be ignored in the model due to their low solubility or low content (less than 0.1%)—pyrite, illite, biotite and other aluminosilicates. The soluble part of the mineral matrix consists mainly of chlorides—NaCl and KCl, other minerals account for less than 0.1%.

According to the present invention, a portion of the specimen (minicore) was selected in a second step to build a three-dimensional model by scanning the specimen by computed tomography. Based on low-resolution scanning of the core in question and measurement of the mineral composition of the sample by scanning electron microscopy, information on the amount and composition of soluble sediments in the sample was obtained. The measurement results are presented in Table 1.

TABLE 1
Geometry

| | | |
|---|---|---|
| Diameter | 29.623 | mm |
| Length | 30.767 | mm |
| External volume | 21.205 | ml |
| Mineral volume | 17.601 | ml |

| | |
|---|---|
| Open porosity | 17.00% |

Composition and volume
of soluble minerals

| | | |
|---|---|---|
| Volume | 2.100 | ml |

| | |
|---|---|
| NaCl content | 88.00% |
| KCI content | 11.97% |

After that, a saline and porosity/permeability characteristic area of the original mineral matrix was selected, from which a sample measuring 8 mm in diameter and 15 mm in length was drilled.

In accordance with the present invention, in a third step, the sample was scanned at a spatial resolution (less than 2.5 μm/voxel) and a three-dimensional model of the sample was constructed, partitioned into insoluble skeleton and soluble sediment, and a representative region of 1000×1000×1000 voxels was selected for which porosity and soluble mineral content matched the original sample.

In accordance with the present invention, in a fourth step, a geochemical model of the mini-core was constructed. When specifying the dissolution reactions, those minerals were not taken into account, which due to their insolubility or small content cannot significantly affect the volume and composition of the system when considering the dissolution of minerals by water. Geochemical modeling was carried out using the chemical simulator Oli Studio (https://www.olisystems.com/oli-studio-scalechem); accordingly, the values of activity coefficients set in it were used.

According to the presented invention, in the fifth step, the numerical modeling (recovery) of the initial saturation of the test sample with water was carried out in several steps. The pore volume of the constructed model was saturated with pure demineralized water and the chemical and thermodynamic equilibrium at reservoir conditions (30° C., 30 MPa) was calculated as described in the relevant section of the proposed invention. Using the Oli Studio chemical simulator (https://www.olisystems.com/oli-studio-scalechem), the volume of dissolved sediment was determined and the pore space of the model was adjusted. After the first simulation step, the error on the total volume of the system under consideration was corrected by increasing the volume of liquid—adding water to the system, after which the equilibrium calculation was repeated. When the values of the error in the balance reached less than the established limit of 0.001 relative volume between the total volume of equilibrium phases and the total volume of open porosity and soluble part of the matrix, the calculation was stopped, and the obtained quantities and composition of liquid and solid phases of the system were considered to correspond to the values under reservoir conditions.

TABLE 2

| Initial | | | After reaching | | |
|---|---|---|---|---|---|
| components | | | equilibrium | | |
| H₂O | KCl | NaCl | Solution | KCl (s) | NaCl (s) |

Step 1. Saturation of the pore volume with water

| | | | | | | |
|---|---|---|---|---|---|---|
| Weight, g | 3.635 | 0.5 | 4.00 | 5.272 | 0.0 | 2.863 |
| Volume, ml | 3.605 | 0.251 | 1.849 | 4.251 | 0.0 | 1.323 |

| | | |
|---|---|---|
| Total, ml | 5.705 | 5.574 |

Step 2. Correction of liquid phase volume (+0.131 ml)

| | | | | | | |
|---|---|---|---|---|---|---|
| Weight, g | 3.767 | 0.5 | 4.00 | 5.452 | 0.0 | 2.815 |
| Volume, ml | 3.736 | 0.251 | 1.849 | 4.401 | 0.0 | 1.301 |

| | |
|---|---|
| Total, ml | 5.702 |

As shown in the dissolution simulation results (Table 2), the total volume of aqueous solution and the remaining solid portion of soluble minerals is 5.702 mL after the second step, which is only 0.003 mL (<0.1%) different from the sum of pore volume (3.605 mL) and soluble minerals (2.1 mL). The obtained composition of the aqueous solution corresponds to the composition of the initial formation water, which is in equilibrium with the rock under reservoir conditions. The properties and composition of the solution calculated from the balance of components are summarized in Table 3.

TABLE 3

| | |
|---|---|
| Volume of solution | 11 |
| H₂O, g | 856 |
| KCl, g | 113.6 |
| NaCl, g | 269.3 |
| Density, g/ml | 1.239 |

In accordance with the present invention, in a sixth step, a sample of a synthetic model of produced water was prepared. Pre-weighed pure salts (potassium chloride 113.6 g and sodium chloride 269.3 g) were placed in a container, to which 856 g of water was then carefully added while stirring with a mechanical stirrer. The resulting solution was then brought to the required thermobaric conditions-reservoir conditions of 30° C. and 300 atm.

1. A method of preparing a formation water model, according to which:
A rock sample containing soluble hard skeletal minerals and/or pore space salt deposits that was initially saturated with formation water and contains at least one area that has not been exposed to drilling fluid is taken from the formation;
The amount and composition of soluble sediments in the collected sample and the water saturation of the sample under reservoir conditions are determined;
At least one internal region in the sample that has not been exposed to drilling fluid is selected;
A construction of a three-dimensional digital model of the selected are a reflecting the pore structure and spatial arrangement of minerals is carried out;
The water solubilization chemical reactions for all water-soluble minerals found in the sample are set;
Numerical modeling of the initial saturation of the sample under study with water is carried out, taking into account the dissolution of minerals included in the sample at the required temperature and pressure until equilibrium is reached;
The final ionic composition of the aqueous solution saturating the pore space of the numerical model after equilibrium is reached is determined;
The solution is prepared by dissolving the calculated number of salts in the calculated amount of water while stirring;
The resulting salt solution is brought to the required thermobaric conditions.

2. The method of preparing a formation water model according to clause 1, wherein selecting an internal region of the sample that has not been exposed to the drilling fluid is based on analyzing the internal structure of the sample obtained by low resolution X-ray tomography.

3. The method of preparing a formation water model according to clause 1, wherein the construction of a three-dimensional digital model of a selected region of the sample is based on a high-resolution X-ray microtomography image.

4. The method of preparing a formation water model according to clause 3, wherein the method additionally uses scanning electron microscopy in imaging modes that provide information about the chemical/mineral composition of the sample under study and the spatial distribution of minerals.
